(19) United States
(12) Patent Application Publication
(10) Pub. No.: US 2018/0044413 A1
(43) Pub. Date: Feb. 15, 2018

(54) Use of MIP-1Beta inhibitors for improving angiogenesis to reduce tissue ischemia and rescue diabetic vascular disease in diabetes mellitus

(71) Applicants: Farmar Licensing Co., Ltd, Taiwan (CN); NATIONAL YANG-MING UNIVERSITY, Taipei (TW)

(72) Inventors: Jaw-Wen CHEN, Taipei (TW); Ting-Ting CHANG, Taipei (TW)

(73) Assignee: NATIONAL YANG-MING UNIVERSITY Taipei Veternas General Hospital

(21) Appl. No.: 15/521,153

(22) PCT Filed: Oct. 23, 2015
(86) PCT No.: PCT/CN2015/092766

§ 371 Date: Apr. 21, 2017

Related U.S. Application Data
(60) Provisional application No. 62/068,475, filed on Oct. 24, 2014.

(51) Int. Cl. C07K 16/24

(52) U.S. Cl. CPC C07K 16/24; C07K 2317/76; C07K 2317/34; A61K 2039/505

(57) ABSTRACT

The present invention relates to a use of MIP-1β inhibitor for promoting angiogenesis in diabetes mellitus patients so as to improve tissue ischemia in damaged area and to prevent diabetic vasculopathy.

### CROSS-REFERENCE TO RELATED APPLICATION

This application claims priority to U.S. Provisional Application No. 62/068,475, filed on Oct. 24, 2014, the entire content of which is hereby incorporated by reference herein in its entirety.

### BACKGROUND OF THE INVENTION

### Technical Field of the Invention

The present invention relates to a use of macrophage inflammatory protein-1β (MIP-1β) inhibitor to reduce tissue ischemia and rescue diabetic vascular disease. Especially, the present invention relates to a use of MIP-1β inhibitor for improving angiogenesis in diabetic patients.

### Background

Vascular complications are the major cause of morbidity and early mortality of diabetic patients, especially in type 2 DM. It is also a common case that diabetic patients are amputated due to the peripheral vascular lesions. There are close links between diabetic vascular diseases and general risk factors of atherosclerosis such as hypertension, high blood lipid and hyperglycemia. These cardiovascular diseases have been increasingly recognized to be vascular inflammation related diseases. However, the treatment efficiency on the cardiovascular disease in diabetic patients is worse than that on the general arteriosclerosis, at present. One of the main reasons is the lack of effective treatment strategy to control the inflammatory responses in diabetic vascular diseases.

Currently, the prevention and treatment effects of vascular disease drugs are mainly focused on the mechanism of controlling general risk factors of atherosclerosis such as blood pressure, blood lipids and blood sugar. These drugs could only delay the onset of diabetes-related vascular disease, or control the disorders after the onset of diabetes-related vascular lesions, which has not been able to control the vascular inflammation of diabetes. Thus, there is a need to seek a new treatment for diabetic vasculopathy by directly regulating the of vascular inflammation diabetes, protecting the blood vessels, enhancing angiogenesis and improving tissue ischemia.

There are certain articles showed that endothelial progenitor cells (EPCs) are essential in neovasculargenesis to maintain the functional and integrity of the vascular endothelial layer. In ischemic tissue, EPCs are also shown to have effects of promoting angiogenesis. The new blood vessels could provide the blood circulation needed in hypoxic tissue, and to reduce the organ damages caused by the lack of oxygen (Asahara, T., et al. EMBO J 18, 3964-3972, 1999; Gallagher, K. A., et al. J Clin Invest 117, 1249-1259, 2007). Therefore, it is also an important subject to find out the way of improving arteriosclerosis and increasing the angiogenesis in hypoxic tissue by promoting the endothelial progenitor cells.

EPC mobilization from the bone marrow compartment is mediated by the SDF-1 receptor CXCR4. And, blockade of CXCR4 on circulating cells prevents progenitor cell recruitment to ischemic sites (Ceradini, D. J., et al. Nat Med 10, 858-864, 2004). In diabetic mice, expression of VEGF and SDF-1 in wound tissue, the number of EPC in peripheral blood, and the expression level of CXCR4 on EPC are decreased (Ceradini, D. J., et al. Journal of Biological Chemistry 283, 10930-10938, 2008).

Macrophage inflammatory protein-1β (MIP-1β, also known as CCL4) is a member of the CC chemokine family that is first isolated from culture medium of LPS-activated macrophage. MIP-1β has a molecular weight (MW) of 7.8 kDa. The protein structure of MIP-1β is constituted as a precursor of 92 amino acids. Mature secreted proteins as 69 amino acids are generated by peptidases that cleave hydrophobic signal peptides. The upregulation of MIP-1β was observed in DM and cardiovascular diseases. In vitro, MIP-1β (also known as CCL4) was able to induce reactive oxygen species production and adhesion of THP-1 cells to human umbilical vein endothelial cells. CCL4 directly induced cell adhesion to endothelial cells through oxidative stress via PI3k-Rac1 cascades (Tatara Y et al., J Mol Cell Cardiol 47:104-111, 2009). Also, CCL4 secreted from macrophages under high glucose condition is capable to induce the endothelial expression of adhesion molecule such as E-selectin in the in vitro study (Chen T C, et al., J Biol Chem 286:25564-25573, 2011).

In the animal model of myocardial infarction, chemokine induction in the infarct heart mediates recruitment of leukocyte subsets with distinct properties. CCL4 and its receptor CCR5 were significantly induced in the infarct mouse myocardium (Dobaczewski M, et al, Am J Pathol 176:2177-2187, 2010). However, the in vivo evidence indicating the direct contribution of CCL4 to vascular and myocardial injury is still lacking.

Thus, we develop a new treatment strategy that increases the proliferation of endothelial progenitor cells, promotes angiogenesis and vessel repair, improves tissue ischemia in diabetic patients by inhibiting the function of macrophage inflammatory protein (MIP)-1β, and further to prevent and treat diabetic vasculopathy and cardiovascular diseases.

### SUMMARY OF INVENTION

The present invention has been found on the above purposes that direct inhibition of macrophage inflammatory protein-1β, such as through the monoclonal antibody and other methods, can produce effects of protecting blood the pancreas function, maintaining insulin secretion, and sopressing the continuous blood vessels in the diabetic animal models.

Accordingly, in one aspect, the present invention features a use of a macrophage inflammatory protein-1β (MIP-1β) inhibitor to prepare a pharmarceutical composition for improving angiogenesis in diabetic subjects. In certain embodiments, the pharmarceutical composition is used to improve tissue ischemia in the diabetic subject. In other embodiments, the pharmarceutical composition is used to prevent vascular lesions in the diabetic subject.

In certain embodiments of the present invention, the MIP-1β inhibitor is a compound capable of decreasing or inhibiting the biological activity of MIP-1β. In one embodiment, the said MIP-1β inhibitor is a MIP-1β-specific ligand, such as an anti-MIP-1β antibody or an antagonist for MIP-1β.

In certain embodiments of the present invention, the anti-MIP-1β antibody is a polyclonal or monoclonal antibody against MIP-1β. In one embodiment, the anti-MIP-1β antibody is a monoclonal antibody, or a protein moiety thereof, having a binding site with binding specificity for a fragment of MIP-1β. In other embodiments, the fragment of MIP-1β comprises an amino acid sequence of ⁴⁶SFVMDYYET⁵⁴ (SEQ ID NO:1) or ⁶²AVVFLTKRGRQIC⁷⁴ (SEQ ID NO:2).

### BRIEF DESCRIPTION OF THE DRAWINGS

FIGS. 1A-1F show MIP-1β inhibition sensitized CXCR4 expression and recovered functions of EPCs from diabetic patients and healthy subjects. CXCR4 expression on EPCs from diabetic patients and healthy subjects (1A; n=6). The migration and angiogenesis abilities in EPCs from diabetic patients (n=5; 1B, 1C). Cell proliferation was measured by MTT and BrdU cell proliferation assay in EPCs from diabetic patients (n=3; 1D, 1E). Western blot and satistical analysis of VEGF and SDF-la expressions in EPCs from diabetic patients (n=3; 1F). #P<0.05, ##P<0.01 compared with untreated basal group. *P<0.05, **P<0.01 compared with MIP-1β 1000 ng/ml alone group.

FIGS. 2A-2B show the inhibitory effects of MIP-1β on neovasculogenesis in normal mice. Color-coded images and blood flow ratio (n=6˜8; 2A, 2B).

FIGS. 3A-3C show the effects of MIP-1β inhibition on EPC homing in ischemia-induced neovasculogenesis in type 1 diabetic mice. Inhibition of MIP-1β activity by monoclonal antibody (mAb) will promote angiogenesis in diabetic mice with hindlimb ischemia operation (OP). Color-coded images and blood flow ratio (n=6-8; A, B). Circulating EPC and Western blot and satistical analysis of VEGF and SDF-la after 2 weeks of MIP-1β neutralizing antibody injection in diabetic mice (n=6; C).

FIG. 4A shows the distribution and amount of the eGFP-CD31 (red fluorescence) double positive cells in the ischemic muscle analyzed by double staining immunohistochemical analysis. FIG. 4B shows the signalling/myofibre ratio. (n=6-8). eGFP positive (with green fluorescence) cell represents bone marrow-derived endothelial progenitor cells. Capillaries were expressed in antibodies directed against CD31 (BD Pharmingen). EPC density was observed by eGFP/CD31 double-positive cells (yellow color) and evaluated by fluorescent microscopy. FIGS. 4C and 4D show the Western blot and statistical analysis of VEGF and SDF-1α after 2 weeks of MIP-1β neutralizing antibody injection in diabetic mice. (n=3). FIGS. 4E and 4F show the CXCR4 expression on peripheral mononuclear cells (E) and bone marrow mononuclear cells (F) (n=6-10). #P<0.05, ##P<0.01 compared with untreated DM mice or control mice.

FIGS. 5A-5F show the effects of MIP-1β neutralization for vessel protection in Lepr^{db/db} diabetic mice. FIGS. 5A and 5B show that inhibition of MIP-1β activity by monoclonal antibody (mAb) will effectively promote angiogenesis in type 2 diabetic mice with hindlimb ischemia operation (OP). Color-coded images and blood flow ratio (n=6˜8; A, B). MIP-1β inhibition increased capillary density in ischaemic muscle than untreated diabetic mice group. FIGS. 5C and 5D are the histograms showing the increased capillary density (C) and the CD31 positive/myofibre ratio after the treatment of anti-MIP-1β monoclonal antibody (mAb) (n=6; C, D). FIGS. 5E and 5F show the circulating EPC (n=6; E) and CXCR4 expression on bone marrow mononuclear cells (n=6; F) in the ischemic limb. #P<0.05, ##P<0.01 compared with untreated DM mice.

FIGS. 6A-6D show the effects of MIP-1β neutralization for vessel protection, promoting ischemia-induced neovasculogenesis, in high fat diet-induced diabetic mice. FIGS. 6A and 6B show the color-coded images (A) and blood flow ratio in the mAb treated diabetic mice after hindlimb ischemia operation (OP) (n=6). FIGS. 6C and 6D show the circulating EPC (n=6; C) and CXCR4 expression on bone marrow mononuclear cells (n=6; D) in the ischemic limb. #P<0.05, ##P<0.01 compared with untreated DM mice.

### DETAILED DESCRIPTION OF THE INVENTION

As used herein, “MIP-1β-inhibitor” refers to a compound that decreases the level of MIP-1β protein and/or decreases at least one activity of MIP-1β protein. In an exemplary embodiment, a MIP-1β-inhibiting compound may decrease at least one biological activity of a MIP-1β protein by at least about 10%, 25%, 50%, 75%, 100%, or more.

In certain embodiments, methods for reducing, preventing or treating diseases or disorders using a MIP-1β-modulating compound may also comprise decreasing the protein level of a MIP-1β, or homologs thereof. Decreasing MIP-1β protein level can be achieved according to methods known in the art. For example, a siRNA, an antisense nucleic acid, or a ribozyme targeted to the MIP-1β can be expressed in or be transfected into the cell. Alternatively, agents that inhibit transcription can be used. Methods for modulating MIP-1β protein levels also include methods for modulating the transcription of genes encoding MIP-1β, methods for destabilizing the corresponding mRNAs, and other methods known in the art.

In other embodiments, the MIP-1β-inhibitor directly or indirectly decreases or inhibits the activity of MIP-1β protein by binding to MIP-1β protein, and thereby to protect pancreas and prevent blood sugar from rising. For instance, according to some embodiments of the present invention, methods for inhibiting the activity of MIP-1β protein in a subject may use an anti-MIP-1β antibody to compete with the MIP-1β protein for binding to its receptor on cell surface. The term “antibody” herein is used in the broadest sense and specifically includes full-length monoclonal antibodies, polyclonal antibodies, multi specific antibodies (e.g., bispecific antibodies), and antibody fragments, as long as they exhibit the desired biological activity.

As used herein, the term “antibody” means an immunoglobulin molecule or a fragment of an immunoglobulin molecule having the ability to specifically bind to a particular antigen. An “antibody fragment” comprises a portion of a full-length antibody, preferably antigen-binding or variable regions thereof. Examples of antibody fragments include Fab, Fab′, F(ab)₂, F(ab′)₂, F(ab)₃, Fv (typically the VL and VH domains of a single arm of an antibody), single-chain Fv (scFv), dsFv, Fd fragments (typically the VH and CH1 domain), and dAb (typically a VH domain) fragments; VH, VL, and VhH domains; minibodies, diabodies, triabodies, tetrabodies, and kappa bodies (see, e.g., Ill et al., Protein Eng 1997; 10: 949-57); camel IgG; and multispecific antibody fragments formed from antibody fragments, and one or more isolated CDRs or a functional paratope, where isolated CDRs or antigen-binding residues or polypeptides can be associated or linked together so as to form a functional antibody fragment.

In certain embodiments, the MIP-1β-inhibitor is a monoclonal antibody specifically binding to the MIP-1β protein. In one embodiment, the anti-MIP-1β monoclonal antibody has the binding specificity for a functional fragment of MIP-1β protein structure. According to some embodiments of present invention, the MIP-1β-inhibitor, such as a monoclonal antibody, binds to the antigen determinant fragment of MIP-1β protein comprising an amino acid sequence of ⁴⁶SFVMDYYET⁵⁴ (SEQ ID NO:1) or ⁶²AVVFLTKRGRQIC⁷⁴ (SEQ ID NO:2).

In some embodiments of the invention, the monoclonal antibody is a humanized antibody or a human antibody.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulations, including (but not limited to) oral compositions such as tablets, capsules, powders and the like, parenteral compositions such as aqueous solutions for subcutaneous, intramuscular or intraperitoneal injection, and lyophilized powders combined with a physiological buffer solution just before administration, are formulated depending upon the chosen route of administration.

The other characteristics and advantages of the present invention will be further illustrated and described in the following examples. The examples described herein are using for illustrations, not for limitations of the invention.

### EXAMPLE

### Example 1. MIP-1β Inhibition Sensitized CXCR4 Expression and Reversed Functions of EPCs

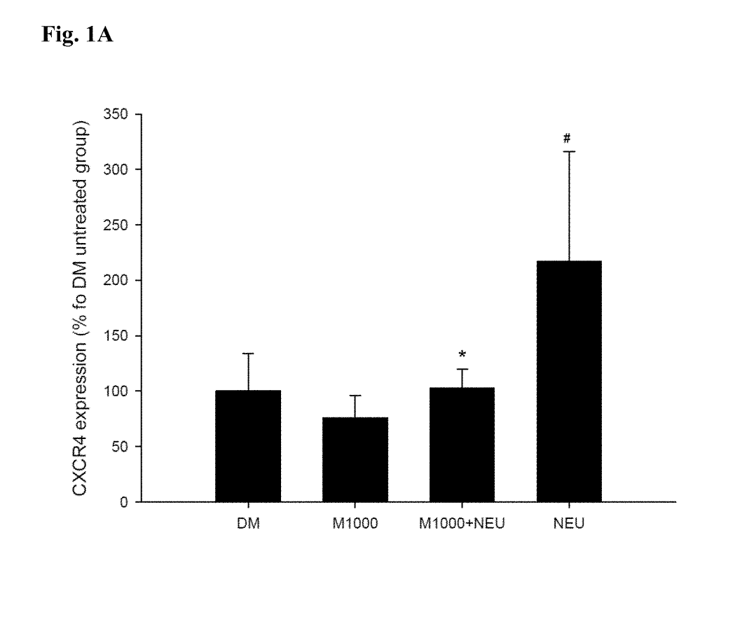

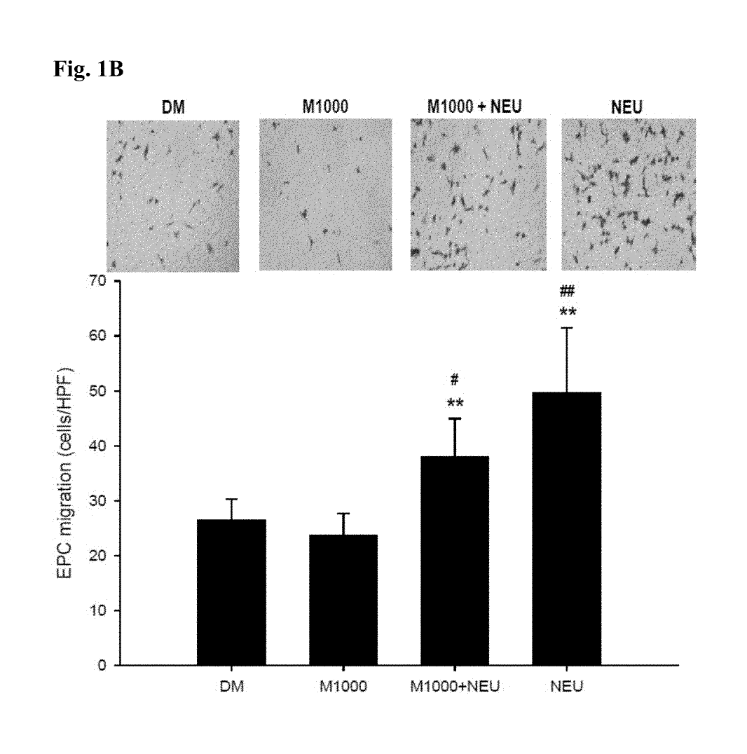

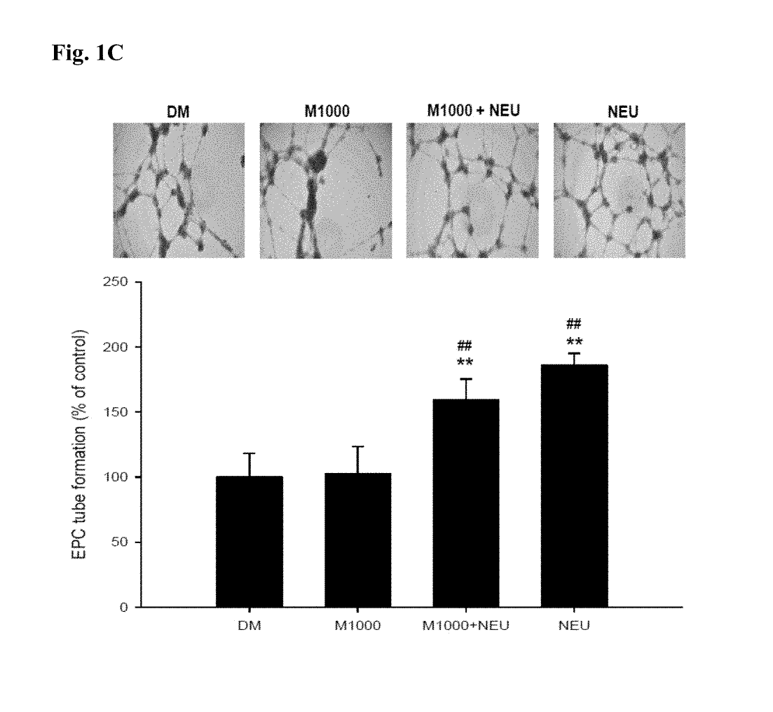

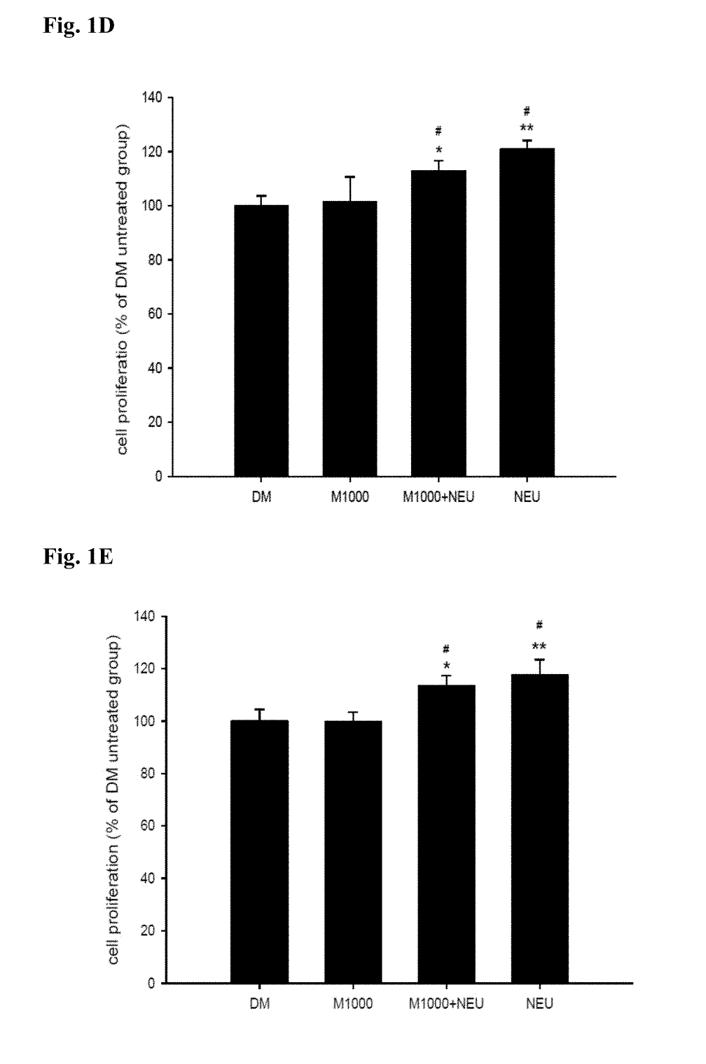

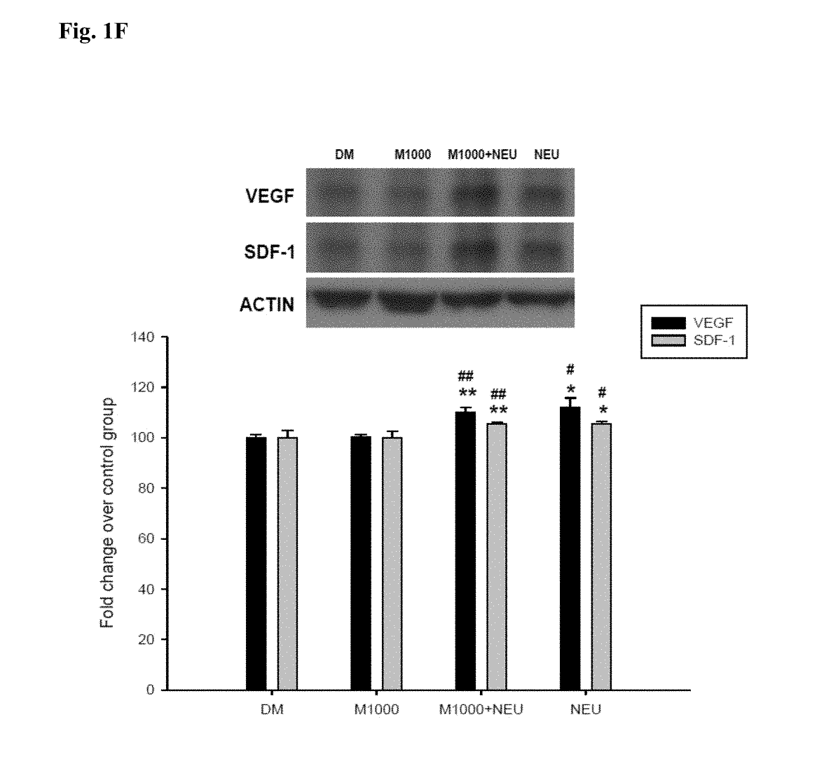

To evaluate the direct effects of MIP-1β and MIP-1β inhibition on EPCs, MIP-1β 1000 ng/ml and MIP-1β antibody were alone or cotreated to EPCs (5×10⁴ cells) from diabetic patients for 30 min. As showed in FIG. 1A, MIP-1β alone did not affect CXCR4 expression, and MIP-1β inhibition significantly increased the CXCR4 level on EPCs.

Thus, we examined if exposure of EPCs to MIP-1β could inhibit their migration toward SDF-1α by a chamber assay. As showed in FIGS. 1B and 1C, the migration toward SDF-1α and in vitro neovasculogenesis abilities of MIP-1β treated EPCs (the M1000 group) were significantly inhibited. On the contrary, the migration and in vitro t neovasculogenesis were recovered in the MIP-1β inhibition groups (the M1000+NEU and NEU groups), as compared to the DM control and MIP-1β treatment (M1000) groups.

Cell proliferation was measured by MTT and BrdU cell proliferation assay in EPCs from diabetic patients. EPC proliferation was enhanced in the MIP-1β neutralizing antibody treated groups (FIG. 1D, 1E). In addition, MIP-1β inhibition could increase the level of VEGF and SDF-1α (FIG. 1F).

### Example 2. MIP-1β Inhibition Protects Blood Vessels in STZ-Induced Diabetic Mice

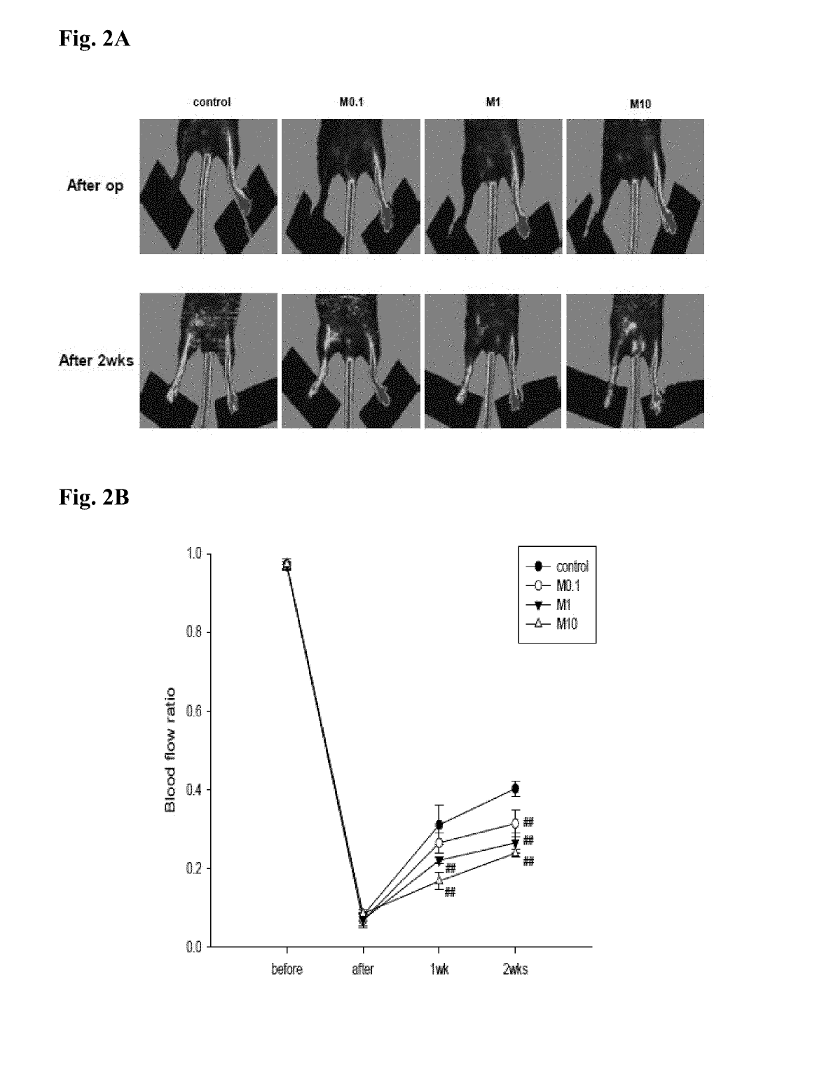

In normal non-diabetic animals, the injection of MIP-1β could inhibit the vascular repair and angiogenesis after hindlimb ischemia operation (OP), reduce the restoration of blood flow in lower limbs, and enhance tissue ischemia (FIG. 2). These suppressive effects of MIP-1β on vessels are dose dependent as the increasing doses of MIP-1β (M0.1 M1.0 μg, M10.0 μg).

Hindlimb ischemia was created in mice that induced to diabetic by STZ injection to confirm the effects of MIP-1β inhibition for enhancing neovasculogenesis in response to tissue ischemia in type 1 diabetic mice. After a 2-week stabilization period, hyperglycemia was generated in 6-week-old male FVB/NJNarl mice by the intraperitoneal injection of streptozotocin (40 mg/kg for 5 days).

The unilateral hindlimb ischemia was induced by excising the right femoral artery. Briefly, the proximal and distal portions of the right femoral artery and the distal portion of the right saphenous artery were ligated. The arteries and all side branches were dissected free and excised.

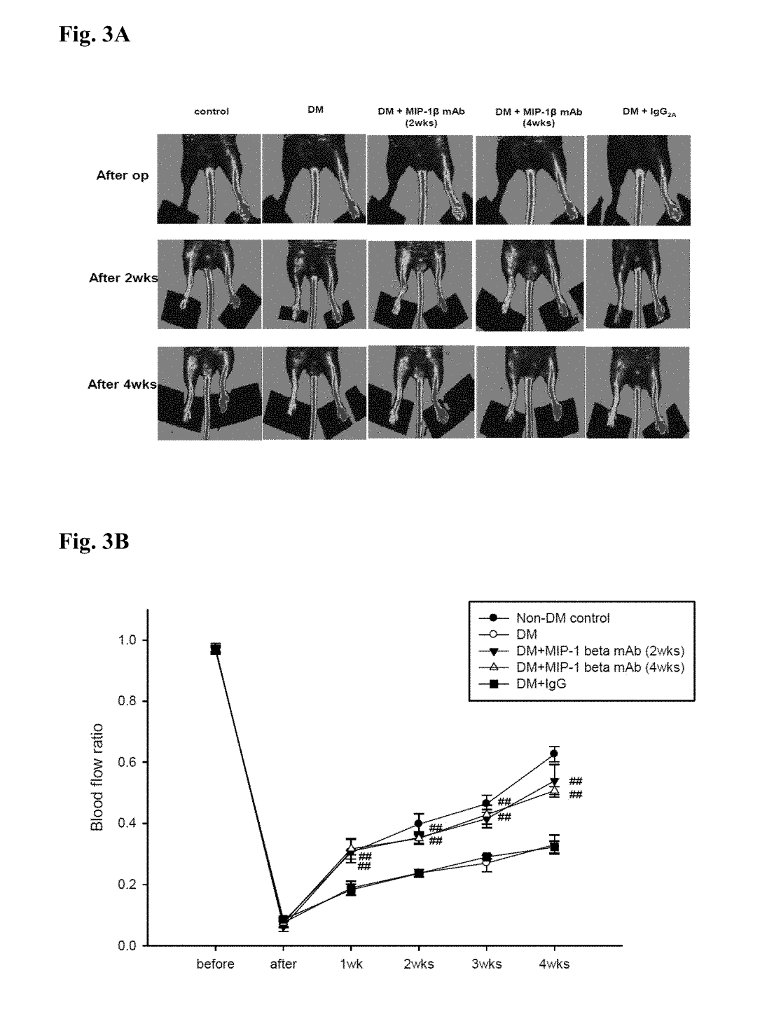

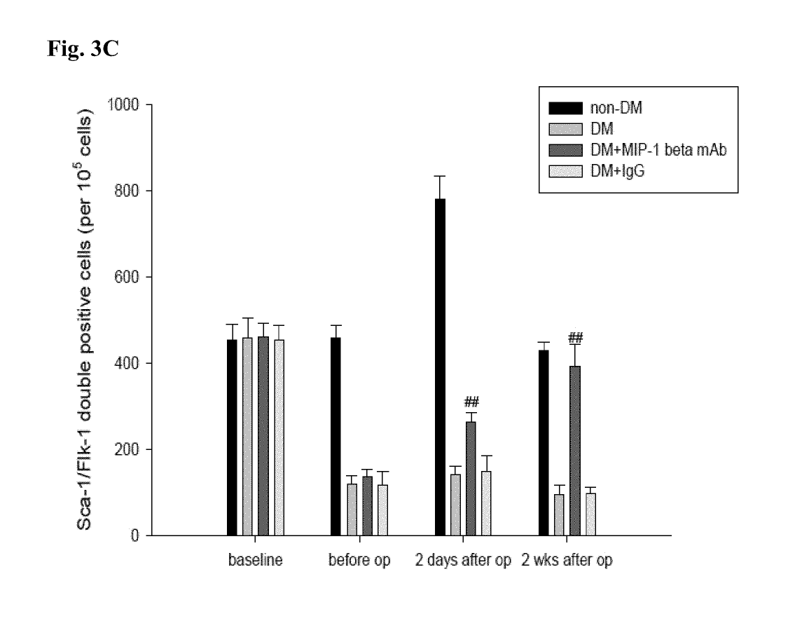

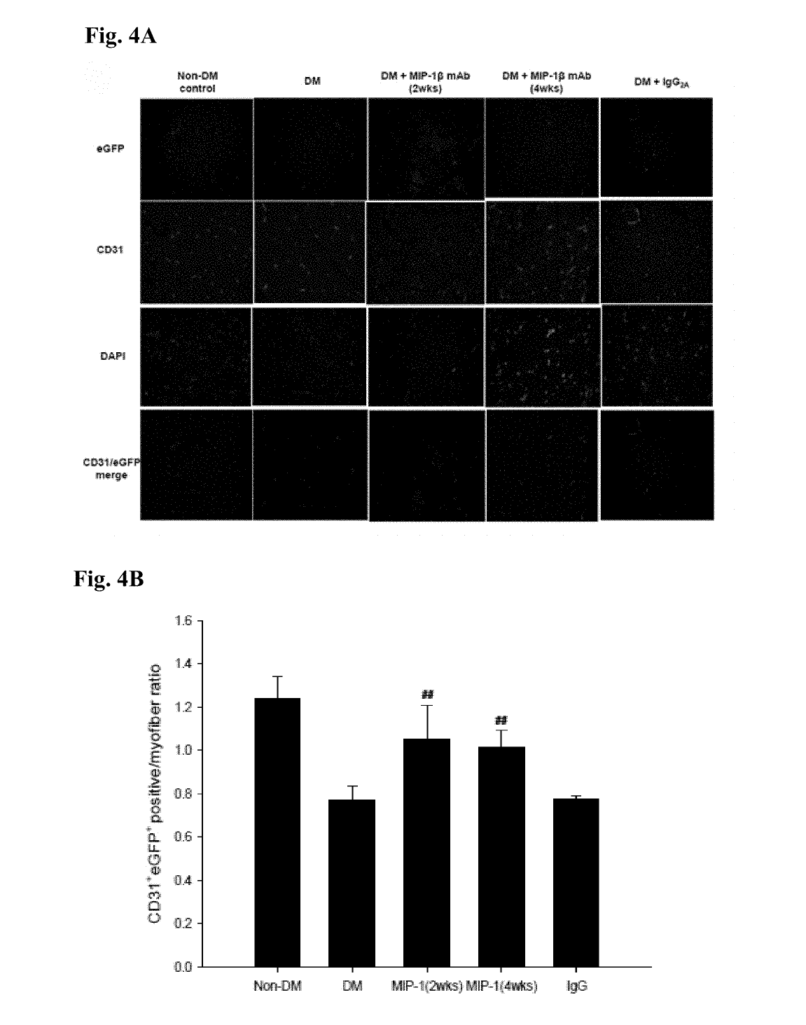

As showed in FIG. 3A, blood flow in the ischemic hindlimb was equally reduced by hindlimb ischemia surgery in each group of mice. Perfusion recovery was markedly attenuated in the diabetic mice compared with non-diabetic ones during the postoperative weeks. The tissue ischemia was notable improved both in the MIP-1β antibody injection for 2 weeks or 4 weeks diabetic mice, and recovered to a similar blood flow ratio as compared with untreated diabetic ones (FIG. 3B).

Furthermore, the increased number of Sca-1⁺/Flk-1⁺ EPC-like cell was attenuated in the untreated diabetic mice group compared with non-diabetic mice at 2 days after ischemic surgery. And, this attenuation of EPC-like cell number was recovered in the MIP-1β antibody injection group compared with untreated diabetic group. The number of EPC-like cells was still increased by MIP-1β inhibition for 2 weeks in diabetic mice (FIG. 3C).

### Example 3. MIP-1β Inhibition Improves Neovasculogenesis by Increasing EPC Homing and Upregulation of VEGF and SDF-1 in Diabetic Mice

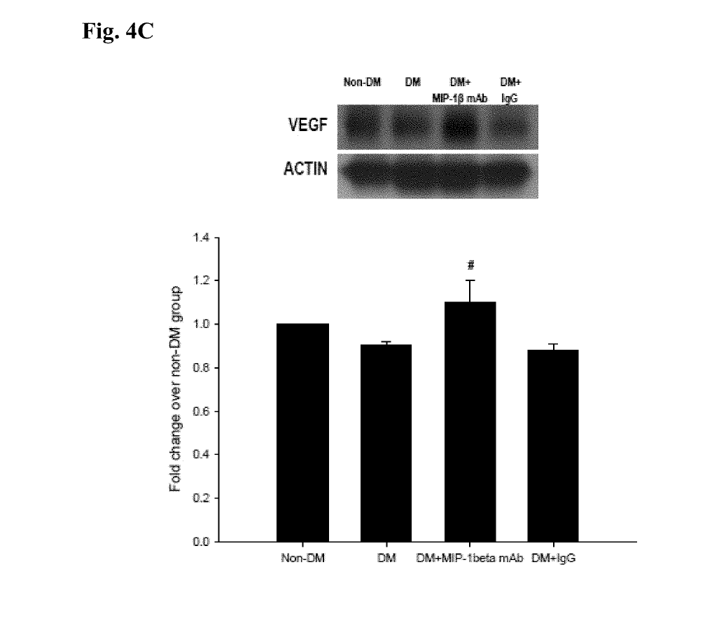

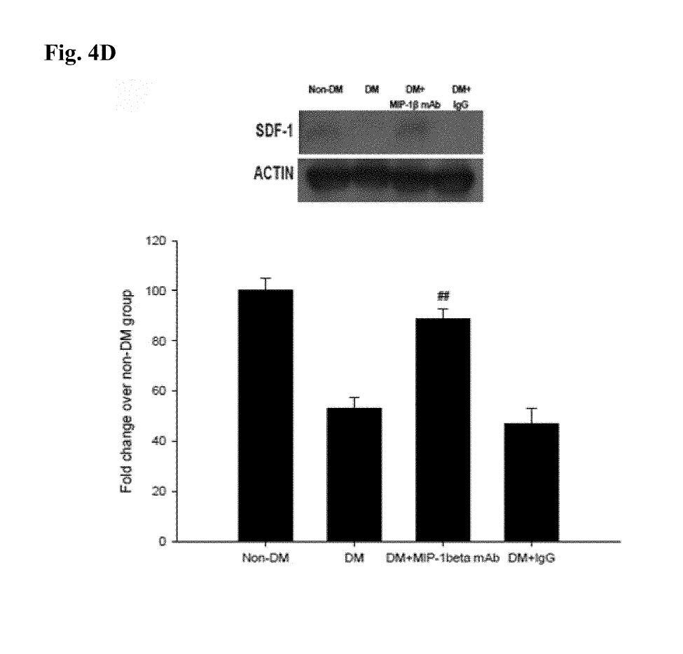

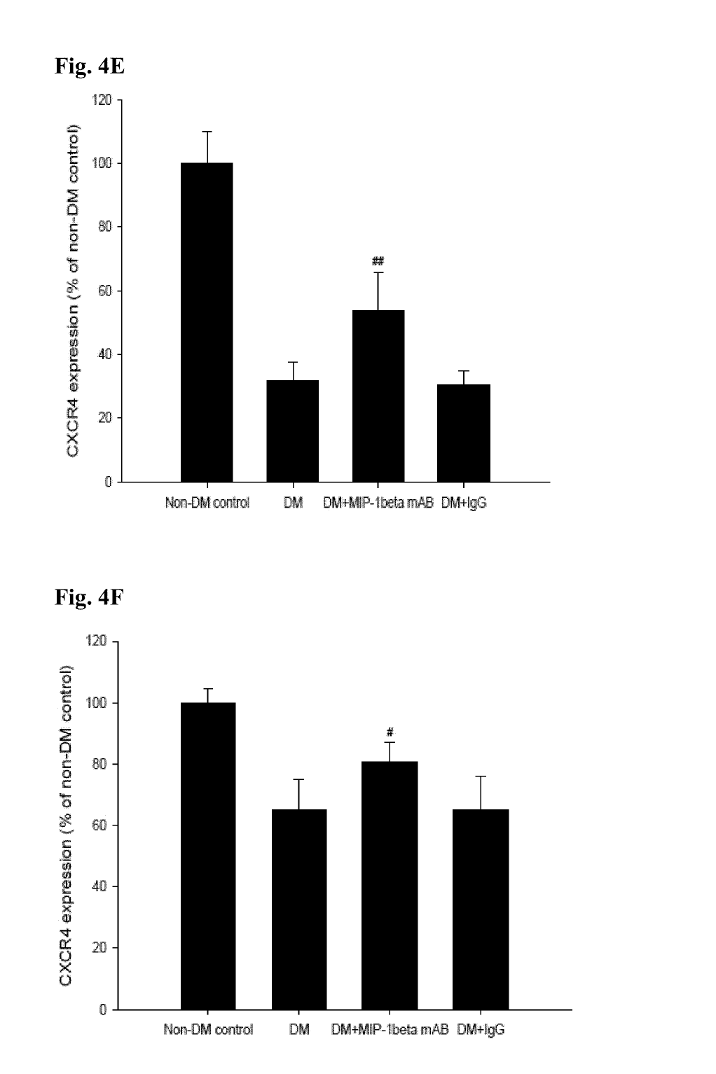

To prove MIP-1β inhibition could improve bone marrow-derived EPC homing, the following in vivo experiments were tested by bone marrow transplantation model. Immunohistochemical analysis revealed that capillary density and homing number of bone marrow-derived EPC in the ischemic limb was reduced in diabetic mice compared with that in non-diabetic mice, and was increased in the MIP-1β antibody treated mice compared with that in untreated mice (FIG. 4A, 4B). The results suggest that MIP-1β inhibition can effectively increase the number of EPC cells (derived from bone marrow) and improve the reduction of capillary vessels in diabetic mice due to tissue ischemia. eGFP positive (green fluorescence) cells represented bone marrow-derived EPCs. Capillaries were expressed in antibodies directed against CD31 (BD Pharmingen) as showed in red color. DAPI staining (blue fluorescence) represents the nucleus EPC density was observed by eGFP/CD31 double-positive cells and evaluated by fluorescent microscopy, showing the cells with capacity of moving to the ischemic position and repairing blood vessel. For the standardized quantitation, the overlapped amount of green and red fluorescence was presented as a ratio with the number of muscle fibers.

And, the expressions of VEGF and SDF-1α in the ischemic muscle were decreased in diabetic mice compared with non-diabetic mice. MIP-1β inhibition group had higher amounts of VEGF and SDF-1α in the ischemic muscle than untreated diabetic ones (FIG. 4C, 4D). MIP-1β inhibition also reversed the decreased CXCR4 expression on peripheral and bone marrow mononuclear cells in diabetic mice (FIG. 4E, 4F).

These findings proved that MIP-1β inhibition could enhance neovasculogenesis in response to tissue ischemia in diabetic mice through increasing EPC-like cell homing, enhancement of VEGF and SDF-1α, and reversed the expression of CXCR4 on peripheral and bone marrow mononuclear cells, which will restore normal blood flow and prevent necrosis in damaged tissue.

### Example 4. MIP-1β Inhibition Protects Blood Vessels and Enhances Ischemia-Induced Neovasculogenesis in Lepr^{db/db} Type 2 Diabetic Mice

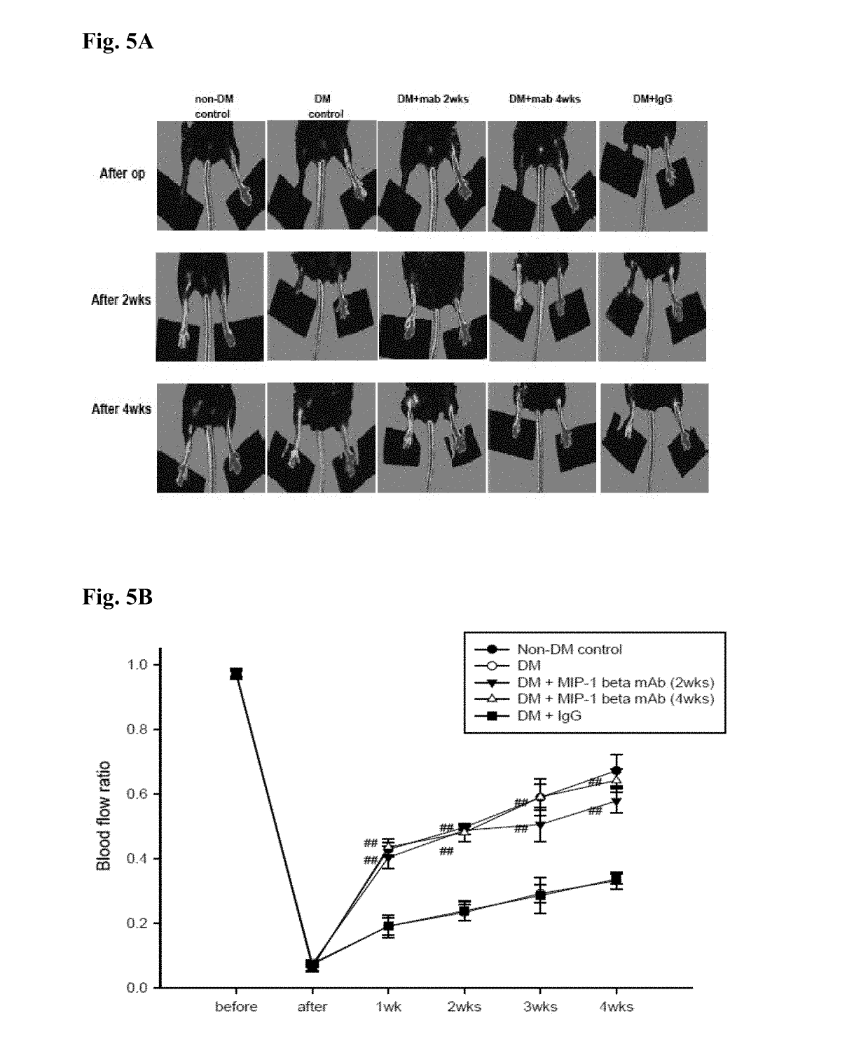

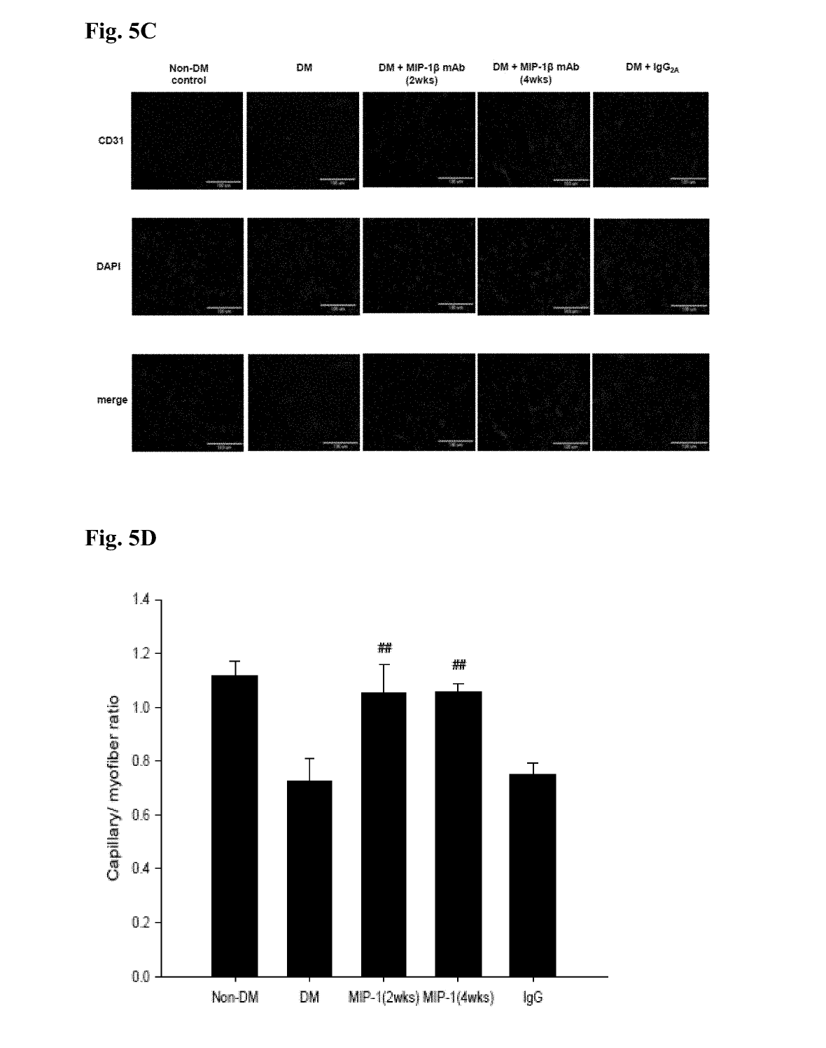

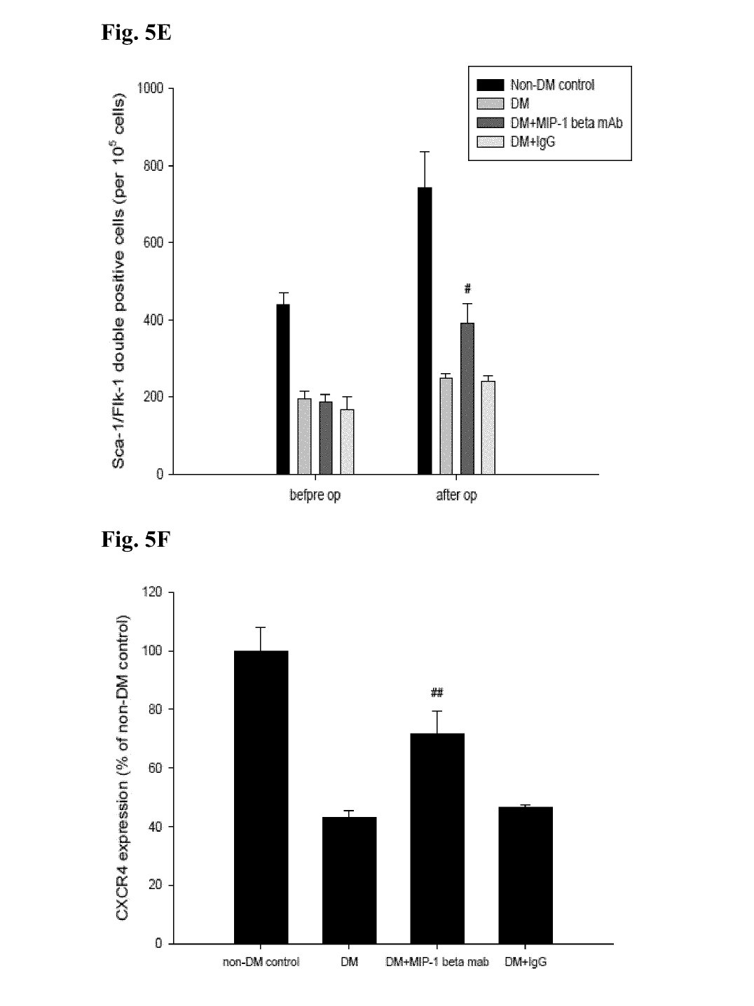

The effects of MIP-1β inhibition in enhancing neovasculogenesis and recovering blood flow of ischemic limbs in type 2 diabetic animals were proved by in vivo tests in Lepr^{db/db} diabetic mice. As the results showed in FIG. 5, MIP-1β neutralization by mAb injection could effectively assist neovasculogenesis after the hindlimb ischemia surgery (OP), increase the recovery of blood flow in hindlimb, and improve tissue ischemia in type 2 diabetic mice (FIG. 5A, 5B).

Similarly, as the results observed in type 2 diabetic mouse model, MIP-1β inhibition enhanced the circulating EPC number in peripheral vessels (FIG. 5C), increased the density of capillary (FIG. 5D), and recovered the bone marrow-derived EPC homing number (FIG. 5E), and restored CXCR4 expression on bone marrow mononuclear cells (FIG. 5F) in diabetic animals with hindlimb ischemia surgery (OP).

### Example 5. MIP-1β Inhibition Protects Blood Vessels and Enhances Ischemia-Induced Neovasculogenesis in High Fat Diet-Induced Diabetic Mice

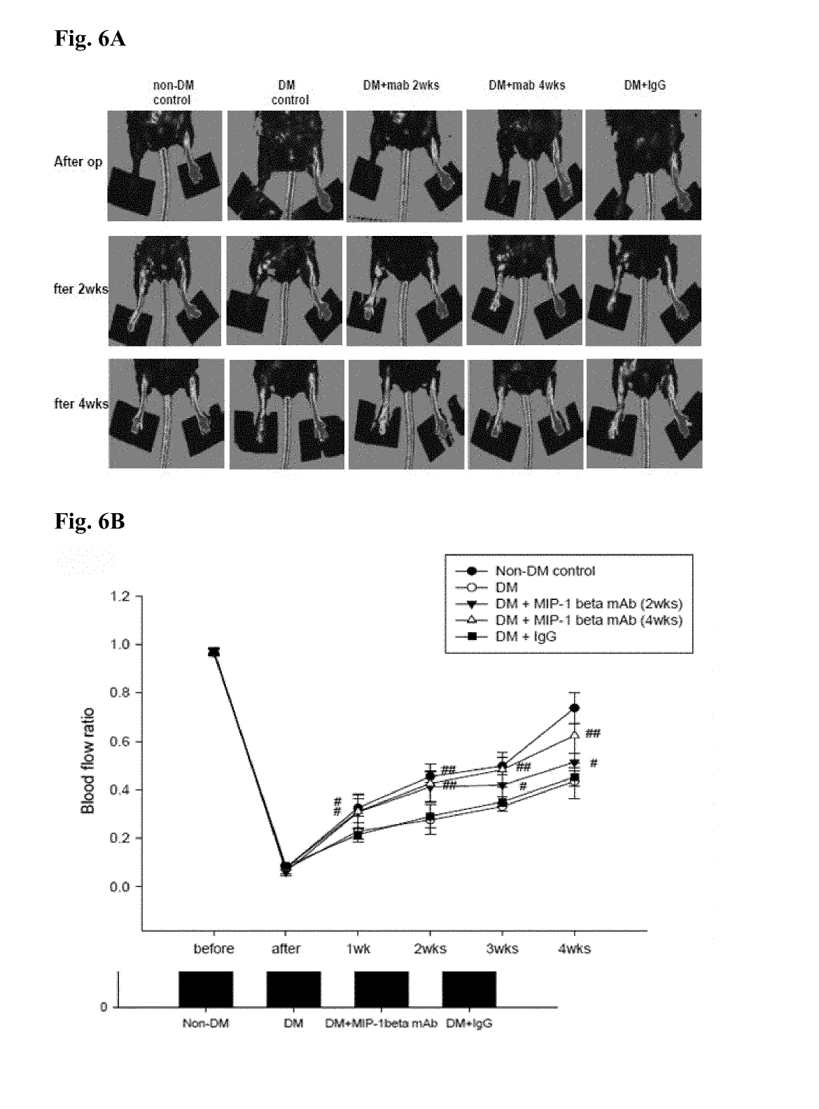

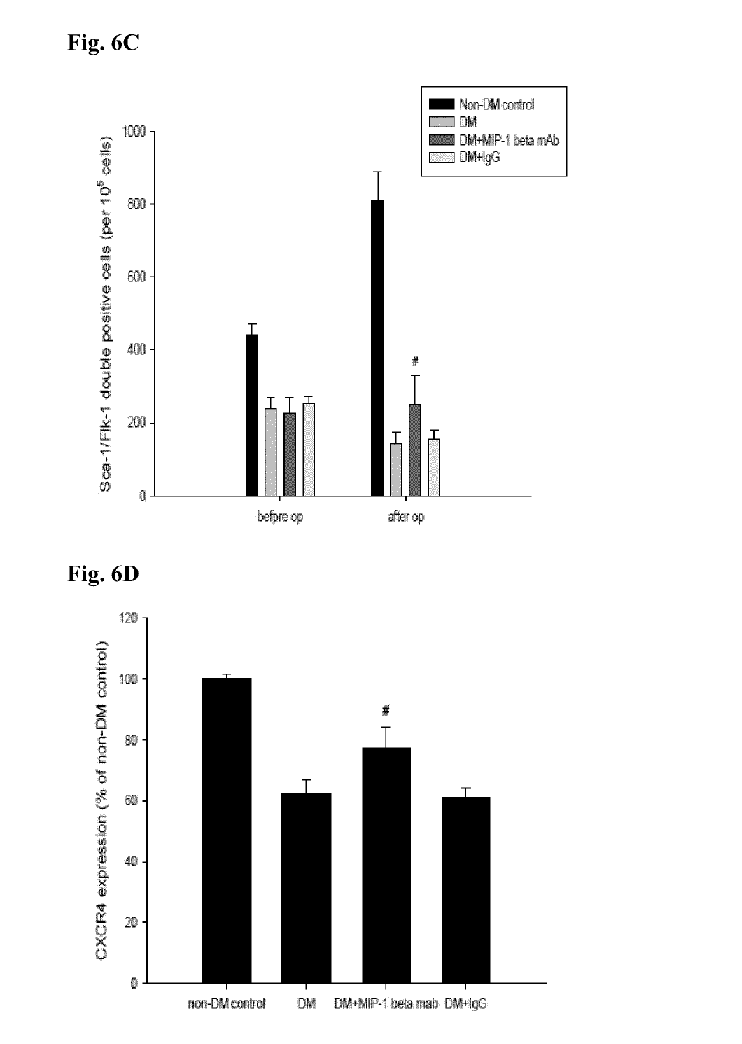

In this example, the effects of MIP-1β inhibition in enhancing neovasculogenesis and recovering blood flow of ischemic limbs in obese diabetic animals were also evaluated in the high fat diet-induced diabetic mouse model. As t showed in FIG. 6, MIP-1β neutralization by mAb injection could effectively assist neovasculogenesis after the hindlimb ischemia surgery (OP), increase the recovery of blood flow in hindlimb, and improve tissue ischemia in the type 2 diabetic mice with high blood sugar level (FIG. 6A, 6B).

In the obese type diabetic mouse model, MIP-1β inhibition could effectively increase the Sca-1+/Flk-1+ EPC-like cell number in peripheral vessels (FIG. 6C), and restored CXCR4 expression on bone marrow mononuclear cells (FIG. 6D) in diabetic animals after the hindlimb ischemia surgery (OP).

These findings showed in the present invention have proved that MIP-1β could suppress neovasculogenesis, and reduce the recovery of blood flow after limb ischemic, aggravating tissue ischemia in normal and diabetic mice. As one embodiment of inhibitory agent for MIP-1β, the MIP-1β neutralizing antibody exhibits effects on diabetic animals, including protecting vessels through increasing EPC-like cell proliferation and homing, enhancing neovasculogenesis, and improving tissue ischemia. Therefore, It has a direct and significant benefit to the prevention and treatment of diabetic vasculopathy by directly inhibiting the in vivo activity and action of macrophage inflammatory protein (MIP)-1β (for example, by administering monoclonal antibody or antagonist against MIP-1β).

1. A use of a macrophage inflammatory protein-1β (MIP-1β) inhibitor to prepare a pharmarceutical composition for promoting angiogenesis in a diabetic subject.

2. The use of claim 1, wherein the pharmarceutical composition is used to improve tissue ischemia in the diabetic subject.

3. The use of claim 1, wherein the pharmarceutical composition is used to prevent the vascular diseases in the diabetic subject.

4. The use of claim 1, wherein the pharmarceutical composition is used to increase endothelial progenitor cells (EPCs) number, repair vascular injury and enhance blood flow recovery after ischemia in the diabetic subject.

5. The use of claim 1, wherein the macrophage inflammatory protein-1βinhibitor is a compound capable of decreasing or inhibiting the biological activity of MIP-1β.

6. The use of claim 1, wherein the macrophage inflammatory protein-1β (MIP-1β) inhibitor is a ligand compound with binding specificity for MIP-1β.

7. The use of claim 6, wherein the MIP-1β inhibitor is an antibody against MIP-1β.

8. The use of claim 7, wherein the antibody is a monoclonal antibody with binding specificity for MIP-1β or a MIP-1β fragment.

9. The use of claim 8, wherein the monoclonal antibody comprises a protein moiety that has a binding site with a fragment of MIP-1θ.

10. The use of claim 8, the monoclonal antibody comprises a binding site for a fragment of MIP-1β comprising an amino acid sequence of SFVMDYYET (SEQ ID NO:1).

11. The use of claim 8, the monoclonal antibody comprises a binding site for a fragment of MIP-1β comprising an amino acid sequence of AVVFLTKRGRQIC (SEQ ID NO:2).


SEQUENCE LISTING (2 sequences)

SEQ ID NO: 1 - protein, 9 aa, Mus musculus
SFVMDYYET

SEQ ID NO: 2 - protein, 13 aa, Mus musculus
AVVFLTKRGRQIC